(19) United States
(12) Patent Application Publication
(10) Pub. No.: US 2013/0079770 A1
(43) Pub. Date: Mar. 28, 2013

(54) IRE GUIDANCE COLLAR

(71) Applicant: PATHFINDER THERAPEUTICS, INC., Nashville, TN (US)

(72) Inventors: ROBERT LYNN KYLE, JR., MURFRESSBORO, TN (US); BENJAMIN WILLIAM NEESE, BRENTWOOD, TN (US)

(73) Assignee: PATHFINDER THERAPEUTICS, INC., NASHVILLE, TN (US)

(21) Appl. No.: 13/624,275

(22) Filed: Sep. 21, 2012

Related U.S. Application Data
(60) Provisional application No. 61/537,696, filed on Sep. 22, 2011.

(51) Int. Cl. A61B 18/14

(52) U.S. Cl. CPC A61B 18/1477
USPC 606/41

(57) ABSTRACT

An IRE guidance collar assembly used to support an IRE needle or other ablation-type devices, comprising a guidance collar eye component and a guidance collar tube component. The collar eye component is movable, and slides freely along the guidance collar tube. This provide rigidity to the IRE needle while allowing various depths of insertion. The IRE needle body is seated in the receiving end of the guidance collar tube, with the needle inserted into and through the tube end of the guidance collar tube. One end of the guidance collar eye component snaps onto and fits over the tube end of the guidance collar tube, while the other end has a needle holder through which the IRE needle is inserted. The collar eye end slides along the tube end of the guidance collar tube, moving the needle holder of the collar end close to or away from the end of the guidance collar tube, as desired.

This application claims benefit of and priority to U.S. Provisional Application No. 61/537,696, filed Sep. 22, 2011, by Rob Kyle, and is entitled to that filing date for priority. The specification, figures and complete disclosure of U.S. Provisional Application No. 61/537,696 are incorporated herein by specific reference for all purposes.

### FIELD OF INVENTION

This invention relates generally to a guidance collar for use with a surgical tool.

### BACKGROUND OF THE INVENTION

Irreversible electroporation (IRE) is a surgical method for treatment of specific cancer types. It is less invasive, can precisely target specific tumors, and has been shown to be a precise and complete method of tumor ablation. IRE induces cell death by creating permanent nanopores in the cellular membrane using high voltage direct electrical current introduced through an IRE tool, such as a needle. These nanopores disrupt the cellular homeostasis.

Success of this surgical method requires accurate placement of the tool tip. A variety of surgical navigation systems involving the tracking of the position of medical instruments or tools during surgical procedures are well known in the art. Examples of such systems, and related tools, are disclosed in U.S. Pat. Nos. 6,190,395 and 7,043,961, the specifications, drawings, and complete disclosures of which are incorporated herein by specific reference for all purposes.

However, IRE needles are very thin, and the flexibility of the needle diminishes the accuracy of the tracking technology. Methods to add rigidity to ablation-type devices typically have been done through use of an introducer cannula. A cannula with a core needle is inserted into the tissue to the desired location of therapy. The core needle is removed, and the ablation device inserted in place of the core. This necessarily involves using a cannula of larger diameter than the ablation device (needle), which can interfere with the therapeutic treatment.

### SUMMARY OF INVENTION

In various embodiments, the present invention comprises an IRE guidance collar assembly used to support an IRE needle or other ablation-type devices. The needle is supported along the majority of its length outside of the patient tissue, thereby avoiding insertion of a cannula into the tissue. This helps the surgeon keep the needle straight while attempting to place it in a specific location prior to delivering therapy, and in showing the needle tip location accurately when used in conjunction with an image-guided surgical system. While described in the context of use with an IRE needle, the invention also can be used with other needles, ablation modalities, and surgical procedures (such as biopsy guidance).

In one embodiment, the IRE guidance collar assembly comprises a guidance collar eye component and a guidance collar tube component. The collar eye component is movable, and slides freely along the guidance collar tube. This provide rigidity to the IRE needle while allowing various depths of insertion. The guidance collar tube retains the collar eye component, although it can be used alone without the collar eye.

In use, the IRE needle body is seated in the receiving end of the guidance collar tube (it can be snapped into place, or secured with a friction fit), with the needle inserted into and through the tube end of the guidance collar tube. One end of the guidance collar eye component snaps onto and fits over the tube end of the guidance collar tube, while the other end has a needle holder through which the IRE needle is inserted. The collar eye end slides along the tube end of the guidance collar tube, moving the needle holder of the collar end close to or away from the end of the guidance collar tube, as desired.

### BRIEF DESCRIPTION OF THE DRAWINGS

FIG. 1 shows a perspective view of an IRE guidance collar assembly attached to an IRE needle.

FIG. 2 shows side and top views of the IRE guidance collar assembly of FIG. 1.

FIG. 3 shows the IRE guidance collar eye component of the IRE guidance collar assembly of FIG. 1.

FIG. 4 shows the IRE guidance collar tube component of the IRE guidance collar assembly of FIG. 1.

### DETAILED DESCRIPTION OF EXEMPLARY EMBODIMENTS

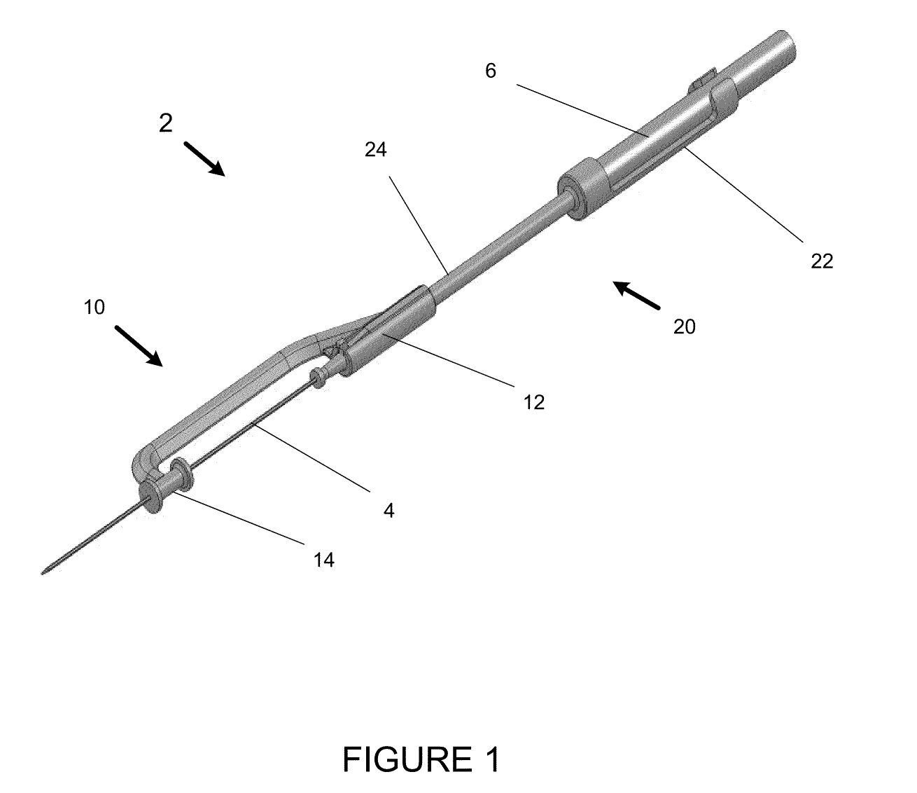

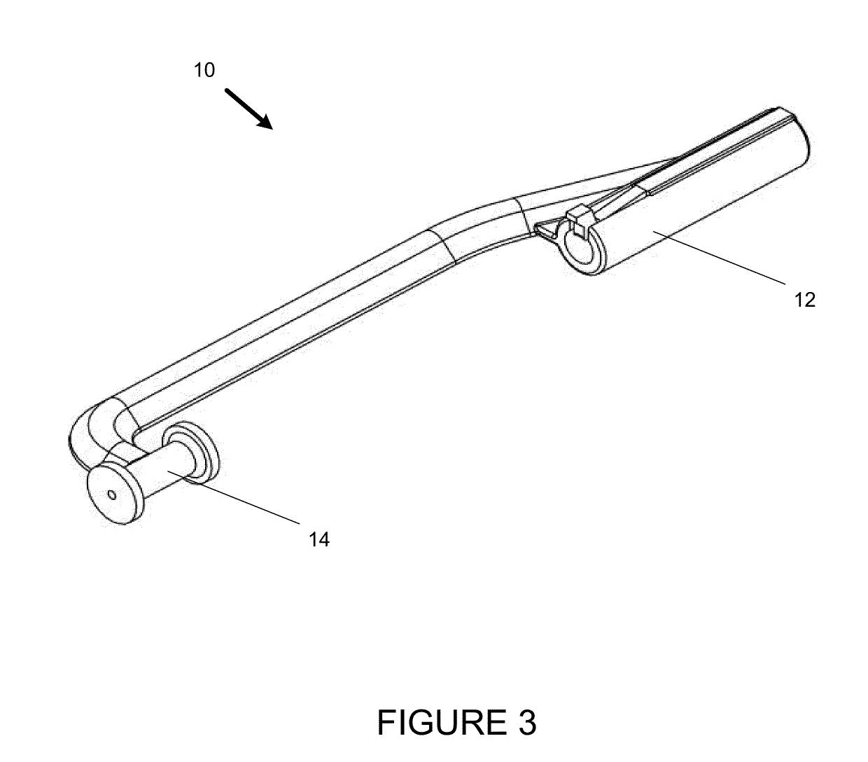

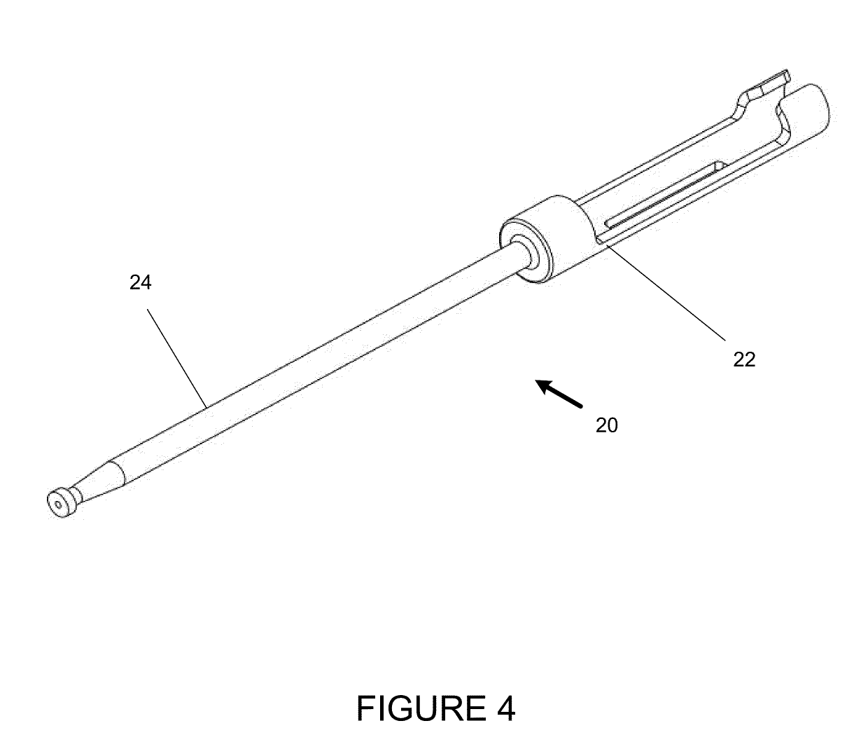

As shown in FIGS. 1-4, the present invention comprises an IRE guidance collar assembly 2 used to support an IRE needle 4 or other ablation-type devices. The needle 4 is supported along the majority of its length outside of the patient tissue, thereby avoiding insertion of a cannula into the tissue. This helps the surgeon keep the needle straight while attempting to place it in a specific location prior to delivering therapy, and in showing the needle tip location accurately when used in conjunction with an image-guided surgical system. While described in the context of use with an IRE needle, the invention also can be used with other needles, ablation modalities, and surgical procedures (such as biopsy guidance).

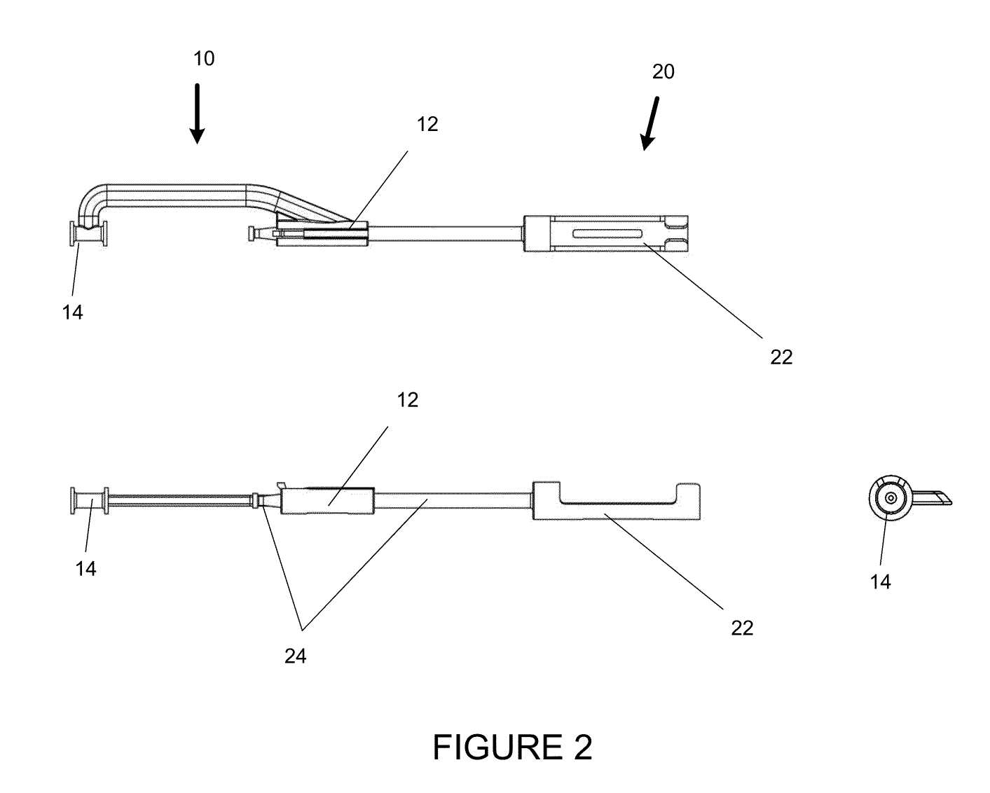

In the embodiment shown in FIGS. 1 and 2, the IRE guidance collar assembly 2 comprises a guidance collar eye component 10 and a guidance collar tube component 20. The collar eye component is movable, and slides freely along the guidance collar tube. This provide rigidity to the IRE needle 4 while allowing various depths of insertion. The guidance collar tube 20 retains the collar eye component 10, although it can be used alone without the collar eye.

In use, the IRE needle body 6 is seated in the receiving end 22 of the guidance collar tube (it can be snapped into place, or secured with a friction fit), with the needle 4 inserted into and through the tube end 24 of the guidance collar tube. One end 12 of the guidance collar eye component snaps onto and fits over the tube end 24 of the guidance collar tube, while the other end has a needle holder 14 through which the IRE needle is inserted. The collar eye end 12 slides along the tube end 24 of the guidance collar tube, moving the needle holder 14 of the collar end close to or away from the end of the guidance collar tube, as desired.

The device thus provides a rigid collar body allowing for improved accuracy of needle placement, while the variable-length free positioning collar eye allows for more accurate and variable depth placement. The device can be constructed and disassembled without requiring the use of tools.

Thus, it should be understood that the embodiments and examples described herein have been chosen and described in order to best illustrate the principles of the invention and its practical applications to thereby enable one of ordinary skill in the art to best utilize the invention in various embodiments and with various modifications as are suited for particular uses contemplated. Even though specific embodiments of this invention have been described, they are not to be taken as exhaustive. There are several variations that will be apparent to those skilled in the art.

What is claimed is:

1. A guidance collar assembly, comprising:
a rigid, hollow guidance collar tube comprising a first and second end, the first end adapted to receive and hold a needle body, wherein a needle extends through the hollow interior of the guidance collar tube and extends from the second end; and
a rigid guidance collar eye component comprising a first and second end, the first end of the guidance collar eye component being slidingly fastened to the guidance collar tube proximate the second end of the guidance collar tube, and the second end of the guidance collar eye component is adapted to hold and support said needle.

2. The assembly of claim 1, wherein the guidance collar eye component is removably fastened to the guidance collar tube.

3. The assembly of claim 1, wherein the second end of the guidance collar eye component is adapted to slide along the length of said needle.

4. The assembly of claim 1, wherein the second end of the guidance collar eye comprises a hole adapted to receive said needle.
